(19) United States
(12) Patent Application Publication
(10) Pub. No.: US 2009/0177080 A1
(43) Pub. Date: Jul. 9, 2009

(54) NONINVASIVE LOCKING OF DISTAL HOLES IN CANNULATED INTRAMEDULLARY NAILS IN SURGERY

(76) Inventors: Anze Kristan, Radavljica (SI); Matej Cimerman, Ljubljana (SI); Paolo Brandoli, Campalto (IT); Tedej Fius, Sevnica (SI); Dario Kreuh, Ljubljana (SI)

Correspondence Address: SMITH MOORE LEATHERWOOD LLP, P.O. BOX 21927, GREENSBORO, NC, 27420, US

(21) Appl. No.: 12/300,944

(22) PCT Filed: Apr. 11, 2007
(86) PCT No.: PCT/SI07/00020

§ 371 Date: Jan. 5, 2009

(30) Foreign Application Priority Data
May 17, 2006 (SI) P-200600123

(51) Int. Cl. A61B 5/05; A61B 17/72

(52) U.S. Cl. 600/424; 606/62

(57) ABSTRACT

A system and a method for facilitating a locking of distal holes in a cannulated intramedullary nail are disclosed. The system includes a transmitter, an internal guide, an extension, and schematic renderings of the distal holes and the extension. The transmitter is capable of representing an origin of a local coordinate system. The internal guide includes a first sensor and is capable of guiding the first sensor along the canal of the cannulated intramedullary nail to the distal holes. The first sensor is capable of transmitting a position and rotation of the internal guide relative to the origin. The extension includes a second sensor and is adapted to allow use of drilling and screwing tools. The second sensor is capable of transmitting a position and rotation of the extension relative to the origin. The schematic renderings of the distal holes and the extension facilitate their alignment along a common axis.

This application is a national stage application under 35 U.S.C. 371 of international application No. PCT/SI2007/000020 filed 11 Apr. 2007, and claims priority to Slovenian Application No. P-200600123 filed 17 May 2006, the disclosure of which is expressly incorporated herein by reference.

The present invention relates to noninvasive locking of distal holes in cannulated intramedullary nails in surgery. In surgical procedures for locking distal holes in cannulated intramedullary nails, invasive and time-consuming methods are used.

The problems of locking of cannulated nails primarily relate to locking of cannulated nails in the distal part of the cannulated nail, which is inserted into the bone, given that in the distal region of the bone the cannulated nail torsionally twists and deviates from its ideal position due to the length thereof and to the individual specifics of the bone canal, making the surgical procedure for accurate locking of cannulated nails lengthy and thus exposing the patient and the surgical staff to a high amount of x-ray radiation. Locking of the cannulated nail in proximal holes is solved by applying angular supports and guides which are anchored to the inserted cannulated nail through a purposely arranged opening and allow for precise drilling and screwing of the screws. Angular supports and external guides are not used for locking distal holes in long bones, requiring longer cannulated nails, because the drilling precision is reduced due to the moment arm being too long and to the torsional twisting which affect the longitudinal deviations and the positioning of the holes in the cannulated nail.

In the field of locking of distal holes in cannulated intramedullary nails in surgery, two prior art methods are defined.

The first method is generally referred to as free-hand locking, wherein the locking of the distal holes is accomplished through the use of C-ARM imaging during the operation. When the intramedullary nail has been positioned in its place inside the bone, locking screws are inserted in the proximal end of the cannulated nail by employing a fixed angular guide. At the distal part, however, locking by means of guides is not efficient, because the cannulated nail gets deformed inside the bone. It may simply bend, but it may also shift torsionally. With the free-hand method, the distal holes must be located by means of an x-ray amplifier. The visual projection of said holes must appear to be perfectly circular, said process of locating said holes by means of x-ray imaging in the course of the operation being time-consuming and lengthy, given that continual readjusting and repositioning of the x-ray head is required in order to achieve an optimal projection of the hole. If the holes appear to be oval or ellipsoidal in shape in any direction, it means that the beam of the x-ray amplifier is not parallel to the holes. When a circular projection of the hole is shown again under the beam of the x-ray amplifier, the surgeon must try to position the drill at the center of the hole, for which the surgeon must be very precise and calm. When the drill is at the center of the hole, it is projected under the x-ray beam as a dot, the drilling gun being aligned parallel to the hole. At this point, drilling may be commenced. In the course of the drilling procedure, the correct alignment must be rechecked several times under the x-ray beam.

It is a disadvantage of this method that both the patient and the surgical staff are exposed to a great amount of radiation. Additionally, the locking of the distal part of the cannulated nail may well represent the lengthiest phase of the operation. On the average, the procedure of locking of the distal part of the cannulated nail takes about 30 minutes.

A second method for locking cannulated nails is an image-guided surgical procedure with navigation. The system consists of transmitters, receivers, software and a computer.

If this method is to be used in the surgery of fractured long bones, a CT scan must be performed prior to the operation of the broken extremity and of the markers which are used in the surgical procedure for calibrating and for accurate positioning of the patient and the CT scans. By this examination, appropriate data are acquired. Said data are then transferred to the computer in the surgery room. In the surgery room, special cameras are disposed on the ceiling for assistance in the positioning of the patient and the markers. Reference points in the form of three-arm guides having a small sphere at the tip of each arm are placed over the injured limb. These points are marked on the CT data as well. All the instruments required for the repositioning of the fracture have markers which are picked up by the camera or the IR emitters. The entire operation is carried out using this method.

It is a disadvantage of the above method that it requires pre-surgery CT imaging of the fractures of the long bones, which is otherwise not usual practice, and, in addition, both the hardware and the software required are very costly and complex. Such surgical procedures may last up to three times longer than free-hand procedures. The method requires the use of many pieces of equipment (cameras, IR emitters). If for whatever reason the system breaks down in the course of the procedure (power outage, software lockup etc.), it is difficult to recalibrate. During the procedure the visual field of the tracers is of vital importance, because the signals are lost when the field of vision is interrupted, which is why the ability to work and the mobility of the surgical staff is limited. The procedure involves lengthy preparatory positioning of the markers inside the patient prior to the CT scanning and also requires the positioning of reference points of the markers prior to the operation, it being possible that due to the robustness of the reference points, the surgeon may inadvertently hit such a reference point, which would then require the reference points to be repositioned and recalibrated anew.

According to the known art, the following problems are not solved: the high amount of x-rays received by the patient and the surgical staff, the lengthiness of the procedure of locking of distal holes, the intricacy of the procedure, and the high complexity of the system.

It is an object of the present invention to achieve a noninvasive locking of distal holes in cannulated intramedullary nails in surgery which will overcome the disadvantages of known and other methods. The present invention evolved from the concrete needs and requests of surgeons to improve on existing procedures, shorten the time needed and ensure greater precision in locking of the cannulated nails. In order for the present invention to fulfill all the said requirements and to exhibit its relative advantages over other systems, the inventive system had to be adapted to the most frequently used, technologically advanced and promising implant. The scope of utilization of the system exceeds the above stated needs, which is confirmed by the fact that, building upon the adaptability and flexibility of the invention, developers and makers of cannulated nails may improve the functionality and complexity of the cannulated nails, thereby improving the applicability and the angular stability and optimizing the fastening thereof The present invention as set forth in the preferred embodiment is targeted at cannulated intramedullary nails and all permutations thereof having a cross-section with a central bore along the entire length thereof.

The object of the invention is achieved with noninvasive locking of distal holes in cannulated intramedullary nails in surgery according to the independent patent claims.

The invention shall now be described according to a preferred embodiment thereof and with reference to the accompanying drawings, showing:

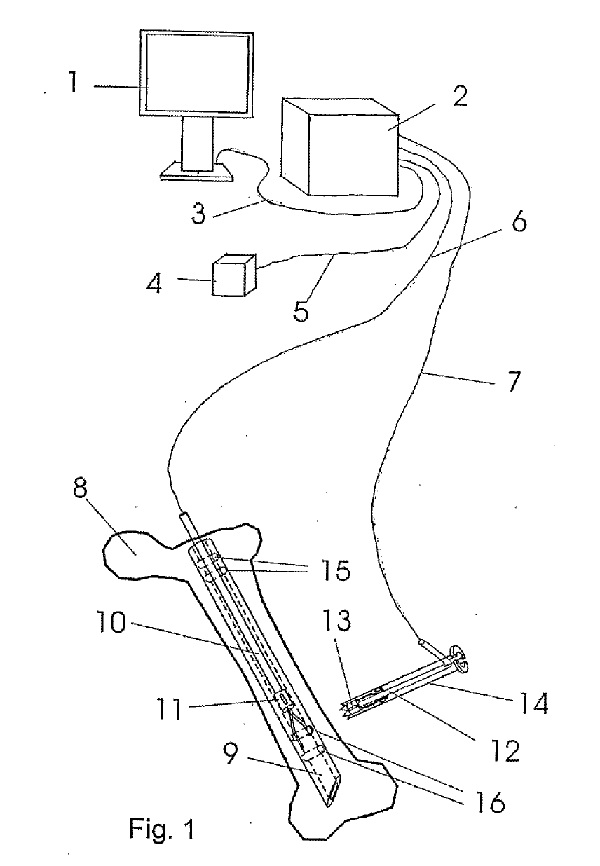

FIG. 1: the system as a whole;

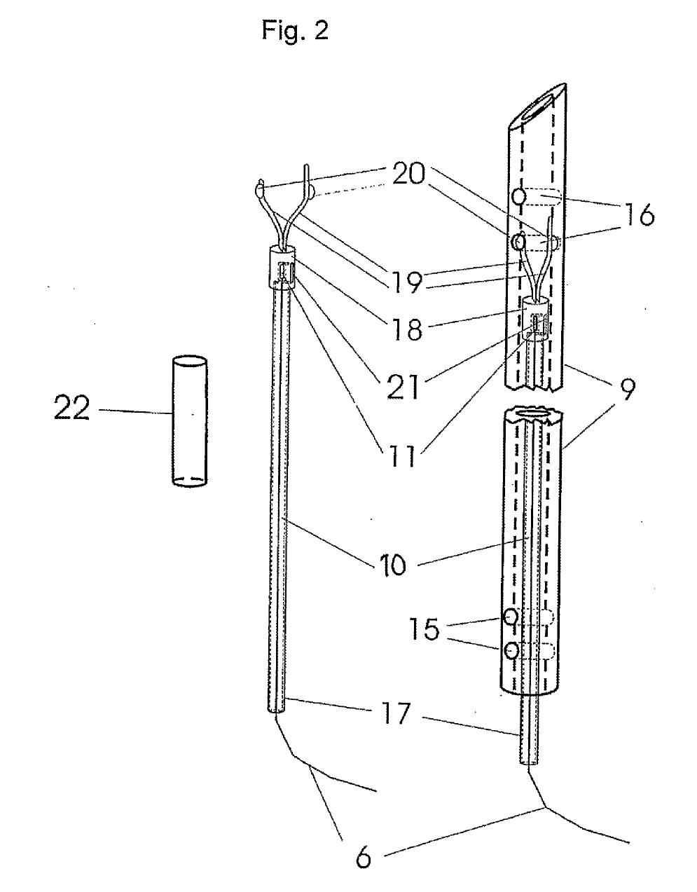

FIG. 2: the internal guide;

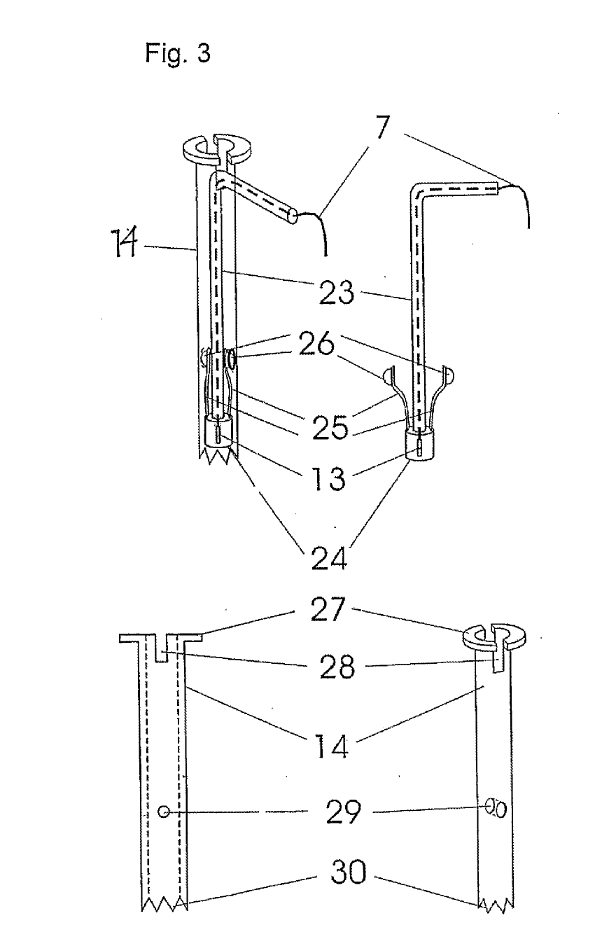

FIG. 3: the external guide;

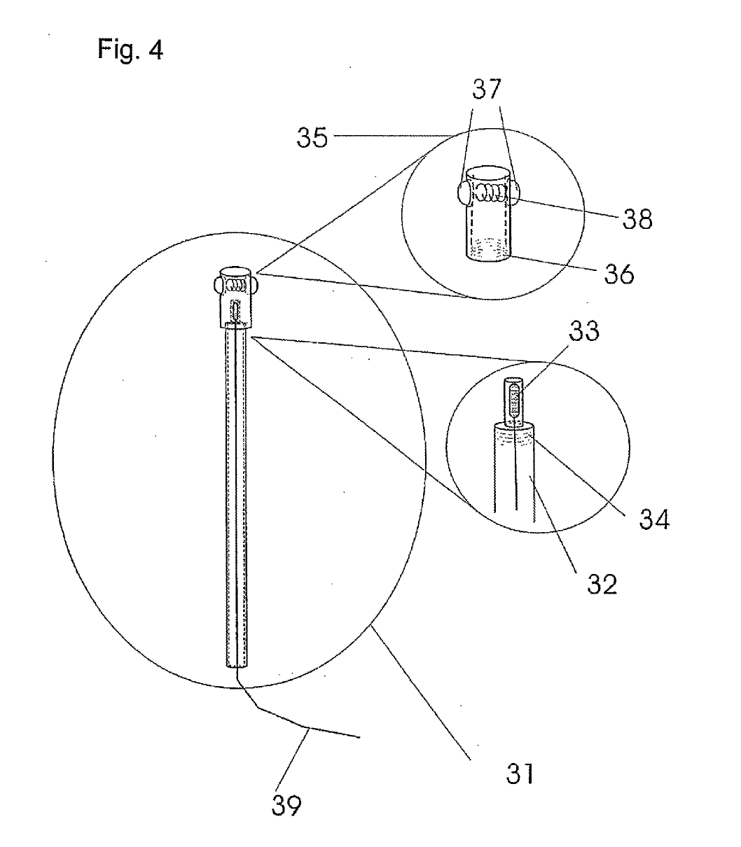

FIG. 4: the modified internal guide;

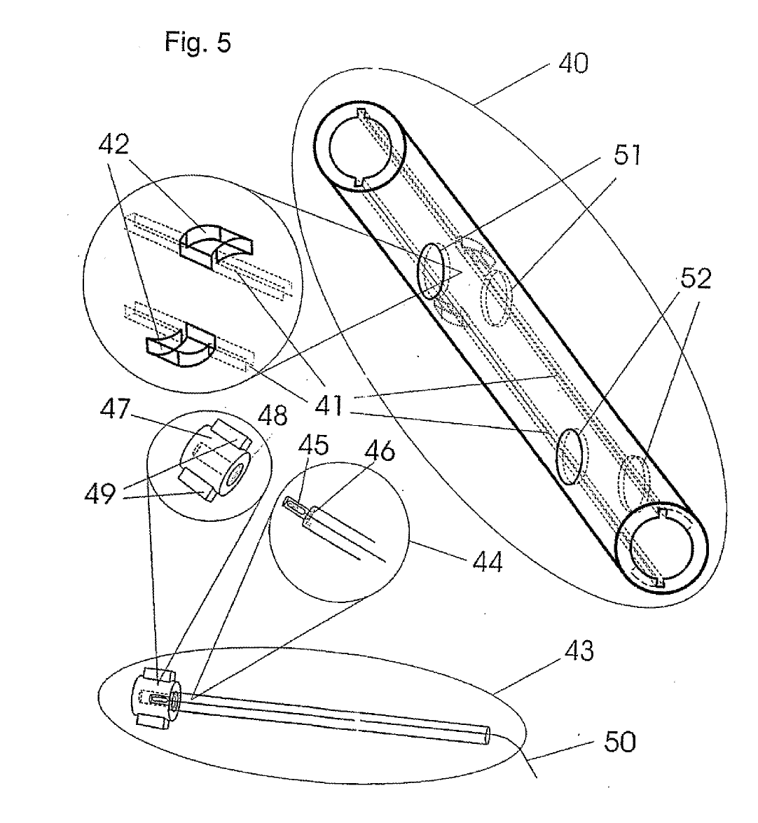

FIG. 5: the cannulated intramedullary nail provided with grooves for inserting the guide with a sensor and the associated guide; and

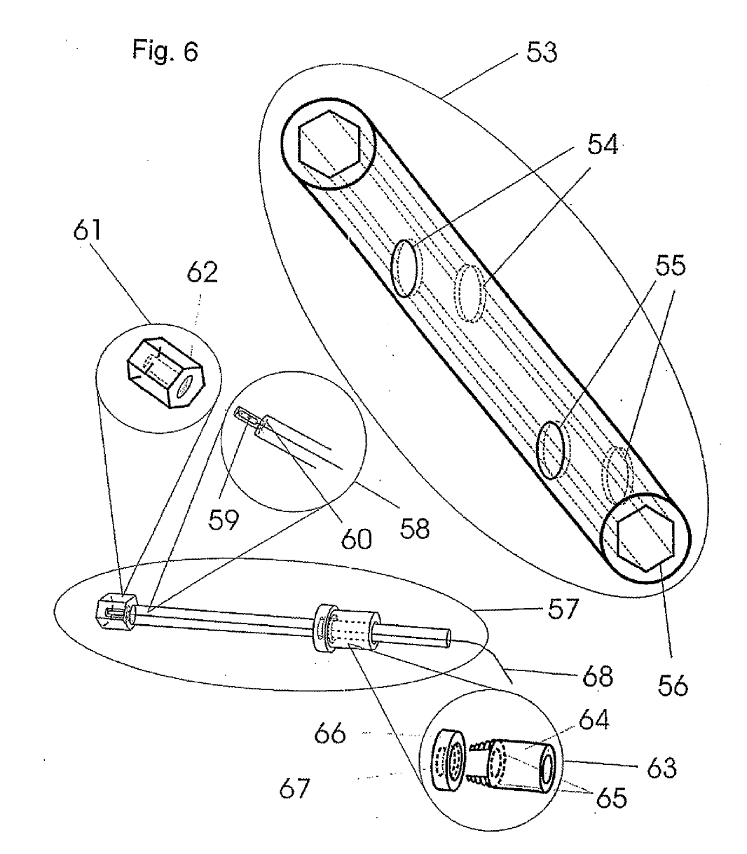

FIG. 6: the cannulated intramedullary nail provided with the shaped-profile canal for inserting the guide with a sensor and the associated guide provided with the shaped-profile canal.

FIG. 1 shows the entire system of the invention for noninvasive locking of distal holes in cannulated intramedullary nails in surgery for repositioning of long-bone fractures, wherein said system is composed of a monitor 1, a computer 2, a VGA cable 3, a transmitter 4, a transmitter cable 5, an internal guide sensor cable 6, an external guide sensor cable 7, an internal guide 10, a sensor 11, an internal guide 10, an external guide 12, an external guide sensor 13, and a tubular extension of the external guide 14.

When the cannulated intramedullary nail 9 is inserted into the long bone 8, the system enables precise, fast, and noninvasive locking of distal holes, given that the internal guide 10 and the sensor 11 are directly inserted into the canal of the cannulated intramedullary nail 9, the internal guide being pushed to the distal holes 16 of the cannulated intramedullary nail 9, where it wedges in. The position and the rotation of the internal guide 10 relative to the origin of the local coordinate system represented by the transmitter 4 is transferred by means of the sensor 11 first to the computer 2, and subsequently to the monitor 1, where it is rendered as a schematic image of the distal hole 16 of the cannulated intramedullary nail 9.

The next step relates to finding the optimal position for drilling and locking of the cannulated nail with self-threading screws. The external guide 12, wherein the sensor 13 of the external guide 12 is located, is inserted into the tubular extension 14 of the external guide 12, wherein it is then moved and rotated manually, the position and rotation thereof relative to the local coordinate system represented by the transmitter 4 being transferred by means of the sensor 13 of the external guide 12 first to the computer 2, and subsequently to the monitor 1, where it is rendered as a schematic image of an arrow. When the schematic image of the distal hole 16 of the cannulated intramedullary nail 9 and the schematic image of the arrow on the monitor 1 are aligned along a common optical axis, the operating phase of locking of distal holes may be started.

FIG. 2 shows the internal guide 10 and its position inside the cannulated intramedullary nail 9. Given the fact that there are various manufacturers on the market, the diameter of the canal in the cannulated intramedullary nails 9 varies and depends upon the individual maker, which is why it was of paramount importance to devise an internal guide 10 that was adaptable to the various diameters of the cannulated intramedullary nail 9. The internal guide 10, an integral part of the system as a whole, is made of a hollow cylindrical tube 17, to which a cylinder with a seating 18 of the sensor 11 is attached. The seating in the cylinder 18 is designed for accommodating the sensor 11, said sensor 11 being secured through the elongated opening 21 on the side of the cylinder 18. On the top of the cylinder with the seating 18 for the sensor 11 two spaced-apart flat lamellae 19 are fixed. The outward curving of the flat lamellae 19 performs the function of a spring and enables adaptation to various diameters of the canal of the cannulated intramedullary nails 9. On the outside of the flat lamellae 19, at the very end thereof, are two spherical nipples 20. The main role and function of the spherical nipples 20 is to engage with the distal holes 16 of the cannulated intramedullary nail 9. The fixed position and rotation of the sensor 11 relative to the position and orientation of the spherical nipples 20 is known in advance and is calculated into the final position and orientation of the distal holes 16 of the cannulated intramedullary nail 9 in the local coordinate system.

The internal guide 10 is inserted through the upper end of the cannulated intramedullary nail 9. Being that the flat lamellae 19 with the spherical nipples 20 are wider than the diameter of the canal of the cannulated intramedullary nail 9, the surgeon must squeeze them together prior to inserting them into the canal of the cannulated intramedullary nail 9. In order for this procedure to be performed as straightforwardly as possible, and in view of the fact that the cannulated intramedullary nail 9 is already driven into the bone, which would make it very awkward for the surgeon to perform said task in vivo through the incision opening, the squeezing of the flat lamellae 19 is performed externally to the open incision and the patient, by means of the tubular extension 22 for inserting the internal guide 10, whereby the flat lamellae 19 are squeezed and, together with the internal guide 10, pushed into the tubular extension 22, which is then in turn pushed into the canal of the cannulated intramedullary nail 9. Following the insertion of the internal guide 10 into the cannulated intramedullary nail 9, the tubular extension 22 for inserting the internal guide 10 is moved aside and driven along the cable 6 of the sensor 11, away from the entrance to the cannulated intramedullary nail 9.

When the internal guide 10 is inserted into the canal of the cannulated intramedullary nail 9, it is rotated by 45 to 90 degrees clockwise relative to the position of the posterior holes 15 of the cannulated intramedullary nail 9, the internal guide 10 being slowly pushed on toward the distal holes 16 of the cannulated intramedullary nail 9. In order for the surgeon to get a better idea of the depth and the approximate position and distance of the internal guide 10 from the distal holes 16 of the cannulated intramedullary nail 9, a millimeter scale is painted on the outside of the hollow cylindrical tube 17 projecting from the canal of the cannulated intramedullary nail 9. These data allow the surgeon better control while inserting the internal guide 10 and approaching the distal holes 16 of the cannulated intramedullary nail 9. When the internal guide 10 approaches the lowest distal hole 16 of the cannulated intramedullary nail 9, the internal guide 10 rotates counterclockwise for a rotational angle from the beginning of the insertion into the canal of the cannulated intramedullary nail 9. In order for the surgeon to get a better idea of how much the guide is rotated, there are longitudinal painted colored lines to assist him, which denote 45-degree angles on the hollow cylindrical tube 17 projecting from the cannulated intramedullary nail 9. After rotating the internal guide 10 back to the approximate position of the holes, the surgeon approaches the position of the lowest distal hole 16 of the cannulated intramedullary nail 9 by making slow back and forth movements and small rotations. Since there is at the end of the internal guide 10 a spring extension consisting of two flat lamellae 19 and two spherical nipples 20, said nipples engage with the distal hole 16 of the cannulated intramedullary nail 9 when the internal guide 10 is in the correct position relative to the existing distal holes 16 of the cannulated intramedullary nail 9. Said engagement with the distal hole 16 of the cannulated intramedullary nail 9 is sensed as a slight jerk at the hollow cylindrical tube 17, which is held by the surgeon's fingers. When the internal guide 10 is placed inside the distal hole 16 of the cannulated intramedullary nail 9, it may no longer be rotated or moved due to the spherical nipples 20 which are conveniently shaped so as to fit very tightly into the distal hole 16 of the cannulated intramedullary nail 9 along the longitudinal axis of the cannulated intramedullary nail 9. If the next distal hole 16 of the cannulated intramedullary nail 9 is to be located, the cylindrical tube 17 must be pulled or rotated a bit stronger, thereby disengaging the spherical nipples 20 and unlocking the internal guide 10. The locked internal guide 10 transmits its position and rotation relative to the local coordinate space with the origin in the transmitter 4 by means of the sensor 11, which is fixed in a special seating of the cylinder 18. The position and rotation of the sensor 11 are transferred via the cable 6 of the sensor 11 to the computer 2 and subsequently as a schematic image of the distal hole 16 of the cannulated intramedullary nail 9 to the monitor 1. The definition of the position and rotation of the distal hole 16 of the cannulated intramedullary nail 9 is thus locked and the surgeon may begin locating the central axis of the distal hole 16 of the cannulated intramedullary nail 9 by means of the external guide 12.

FIG. 3 shows the external guide 12 and the tubular extension 14 of the external guide 12 in their functional connection, wherein the external guide 12 and the tubular extension 14 of the external guide 12 are envisioned to allow surgeons to employ standard tools in the drilling and screwing procedures.

The external guide 12, which is an integral part of the system as a whole, is made of an angularly bent hollow cylindrical tube 23, to which a cylinder with the seating 24 of the sensor 13 of the external guide 12 is fixed. The seating in the cylinder 24 is provided for the sensor 13 of the external guide 12, wherein the sensor 13 of the external guide 12 is secured. The position and rotation of the sensor 13 of the external guide 12 are transferred via the cable 7 of the sensor 13 of the external guide 12 to the computer 2 and subsequently, as a schematic image of an arrow, to the monitor 1. At the bottom side of the cylinder with the seating 24 of the sensor 13 of the external guide 12, two spaced-apart flat lamellae 25 of the external guide 12 are fixed. The outward curving of the flat lamellae 25 of the external guide 12 performs the function of a spring. The flat lamellae 25 of the external guide 12 are facing in the opposite direction of the flat lamellae 19 of the internal guide 10, so that the sensor 13 of the external guide 12, located in the cylinder 24 of the external guide 12, may come as near as possible to the surface, under which the distal hole 16 of the cannulated intramedullary nail 9 is located. On the outside of the flat lamellae 25 of the external guide 12, at their very end, there are two spherical nipples 26 of the external guide 12. The main role and function of the spherical nipples 26 of the external guide 12 is to engage with the fixing holes 29 of the external guide 12 on the tubular extension 14 of the external guide 12. The fixed position and rotation of the sensor 13 of the external guide 12 relative to the position and orientation of the spherical nipples 26 of the external guide 12 is known in advance and is calculated into the final position and orientation when locating the central axis of the distal holes 16 of the cannulated intramedullary nail 9 in the local coordinate system.

The tubular extension 14 of the external guide 12 is provided with a flat edge 27 at the upper end thereof, its purpose being to facilitate hammer blows. A longitudinal groove 28 is made in the flat edge 27 of the tubular extension 12 along the body of the tubular extension 14 of the external guide 12, into which groove 28 the angularly bent hollow cylindrical tube 23 of the external guide 12 is inserted, said groove 28 being deep enough so that the angularly bent hollow cylindrical tube 23 is not damaged by the hammer blows applied for fixing the tubular extension 14 of the external guide 12. In the lower part of the tubular extension 14 of the external guide 12, there are two fixing holes 29 having a diameter that is long enough to allow for the locking of the spherical nipple 26 of the external guide 12. The lower part of the tubular extension 14 of the external guide 12 has a serrated edge 30, allowing the surgeon to secure it to the bone once the optimal position in relation to the central axis of the distal holes 16 of the cannulated intramedullary nail 9 has been found. Before proceeding to locate the central axis of the distal holes 16 of the cannulated intramedullary nail 9, the schematic image of which is rendered on the monitor 1, the surgeon must insert the external guide 12 into the tubular extension 14 of the external guide 12, making sure that the spherical nipples 26 of the external guide 12 are locked in the fixing holes 29 of the tubular extension 14 of the external guide 12 and also that the angularly bent hollow cylindrical tube 23 is locked in the longitudinal groove 28 of the tubular extension 14 of the external guide 12. When said conditions are met, the tubular extension 14 of the external guide 12 with the inserted external guide 12 behaves as one entity. The procedure is carried on by moving the tubular extension 14 of the external guide 12 over the surface of the patient's skin until it takes up the correct position relative to the central axis of the schematic image of the distal holes 16 of the cannulated intramedullary nail 9 as displayed on the monitor 1. The motion and rotation of the tubular extension 14 of the external guide 12 is rendered in real time on the monitor 1 as the schematic image of an arrow, thus allowing for very straightforward manipulation.

When the most appropriate position for the tubular extension 14 of the external guide 12 is found, an opening is incised in the skin and the muscle mass is moved aside in order to enable the tubular extension 14 of the external guide 12 to reach the bone. On the bone, further fine manipulation i.e. more precise positioning of the tubular extension 14 of the external guide 12 is carried out. Once the position of the tubular extension relative to the distal hole 16 of the cannulated intramedullary nail 9 is optimal, the fixation procedure is performed. The surgeon strikes the flat edge 27 of the tubular extension 14 of the external guide 12 with a hammer, securing the serrate edge 30 of the tubular extension 14 of the external guide 12 into the bone. Once the fixation is completed, the external guide 12 is extracted from the tubular extension 14 of the external guide 12. The internal guide 10, located in the cannulated intramedullary nail 9 and locked in the lowest distal hole 16 of the cannulated intramedullary nail 9, is extracted to the next distal hole 16 of the cannulated intramedullary nail 9, where it awaits the next phase, when the lowest distal hole 16 of the cannulated intramedullary nail 9 is locked by a screw and the tubular extension 14 of the external guide 12 is removed, to be arranged for another positioning.

FIG. 4 shows a modified guide 31 with a sensor 33 as one of many possible modifications of a guide with a sensor which is inserted into the cannulated intramedullary nail 9 for noninvasive locking of distal holes. It is mainly a guide whose functionality is generally similar to that of the internal guide 10 or the external guide 12 and does not exhibit any essential modifications, the main modification being in the general concept of the embodiment as such, namely, the guide being constructed in such a way that it can be utilized both as an internal guide 10 and as an external guide 12. The modified guide 31 with the sensor 33 is composed of a hollow cylindrical tube with a seating 32 for the sensor 33 of the modified guide 31, said hollow cylindrical tube 32 having on its upper side a thread 34 for fastening a spacer cylinder 35, a sensor 33 of the modified guide 31, a spacer cylinder 35 having an inner thread 36 on the bottom side thereof for mounting the hollow cylindrical tube 32, spherical nipples 37 of the spacer cylinder 35, a spring 38 and a cable 39 of the sensor 33 of the modified guide 31. Thanks to the modified guide 31 with the sensor 33, the procedure of the locking of the distal holes 16 of the cannulated intramedullary nail 9 differs in the initial phase when insertion into the cannulated intramedullary nail 9 takes place, in that no tubular extension 22 is required. The rest of the procedure of the locking of the distal holes 16 of the cannulated intramedullary nail 9 is identical to the one described with reference to FIG. 2, with the difference that the function of the spring, performed by the two flat lamellae 19 and 25, is now transferred from the internal guide 10 and the external guide 12 to the inserted spring 38 of the spacer cylinder. Thus, the spherical nipples 37 of the spacer cylinder 35 give in, depending on the diameter of the canal of the cannulated intramedullary nail 9, while the modified guide 31 travels along the canal. When the modified guide 31 with the sensor 33 hits the distal holes 16 of the cannulated intramedullary nail 9, the spherical nipples 37 of the spacer cylinder 35 are locked by the distal holes 16 due to the action of the spring 38, which pushes them apart. The remaining functionality and the procedure of locking as such are identical to the functional description of the internal guide 10 and the external guide 12, including the transmission of the position and rotation of the sensor 33 via the cable 39 of the sensor 33 of the modified guide 31 to the computer 2 and subsequently as a schematic image to the monitor 1.

FIG. 5 represents a possible embodiment for noninvasive locking of distal holes 51 from the manufacturers' perspective, wherein a manufacturer develops and produces a cannulated intramedullary nail 40 with preformed grooves 41 for inserting an appropriately shaped guide 43 with a sensor 45. The cannulated intramedullary nail 40 is essentially a conventional cannulated intramedullary nail 9 with preformed grooves 41 inside its canal, which grooves enable the inserting and guiding of the appropriately shaped guide 43 to the distal hole 51, where the appropriately shaped guide 43 is stopped and locked by the positional groove 42, thereby allowing the sensor 45 of the appropriately shaped guide 43 to transmit the position and rotation of the distal hole 51 via the cable 50 of the sensor 45 of the appropriately shaped guide 43 to the computer 2 and subsequently to render the distal holes 51 in a schematic manner on the monitor 1. The appropriately shaped guide 43 according to the said embodiment is composed of a hollow cylindrical tube 44 with the seating of the sensor 45 of the appropriately shaped guide 43 having at its upper end a thread 46 for fixing the cylinder 47 with spacer locks 49, the sensor 45 of the appropriately shaped guide 43, which is connected to the computer 2 via the cable 50 of the sensor 45 of the appropriately shaped guide 43, and the cylinder 47 with the spacer locks 49, having at its inner lower end an internal thread 48 for fixing to the hollow cylindrical tube 44. The procedure of the locking of the distal holes 51 of the cannulated intramedullary nail 40 with preformed grooves 41 for inserting of the appropriately shaped guide 43 with the sensor 45 and of the appropriately shaped guide 43 does not differ from the above mentioned procedures as described with reference to FIGS. 1, 2, 3 and 4, in that, as far as functionality is concerned, only the embodiment of the guides and of the cannulated intramedullary nail is modified, which does not affect the procedure of locking of the cannulated nails as such. The essential advantage of the said embodiment is in that, in accordance with the procedure of locking of the distal holes 51 as set forth in the present invention, the manufacturer of cannulated nails may start producing even better and higher quality cannulated nails that will be better adapted to the specifics of the fractures of long bones and to the locking thereof according to the noninvasive method.

FIG. 6 shows an embodiment of noninvasive locking of distal holes 54 from the perspective of the manufacturers of cannulated nails, wherein the manufacturer develops and produces a cannulated intramedullary nail 53 having a shaped-profile canal 56, the said canal of the cannulated intramedullary nail 53 having an arbitrary profile which may be shaped as an ellipse, a triangle, a square, a hexagon, or a higher polygon and into which a matching shaped-profile guide 57 is inserted, which shaped-profile guide must match the shaped-profile canal 56. The cannulated intramedullary nail 53 is essentially a conventional cannulated intramedullary nail 9 having a shaped-profile canal 56 that enables the inserting and guiding of the matching shaped-profile guide 57 to the distal hole 54, where the matching shaped-profile guide 57 is stopped by the external spacer stopper 63, thereby enabling the sensor 59 of the matching shaped-profile guide 57 to transmit the position and rotation of the distal hole 54 via the cable 68 of the sensor 59 of the matching shaped-profile guide 57 to the computer 2 and subsequently to render the distal holes 54 in a schematic manner on the monitor 1. The matching shaped-profile guide 57 according to the said embodiment is composed of a hollow cylindrical tube 58 with the seating of the sensor 59 of the matching shaped-profile guide 57, having at its upper end a thread 60 for fixing the shaped-profile cylinder 61, the sensor 59 of the matching shaped-profile guide 57, which is connected to the computer 2 via the cable 68 of the sensor 59 of the matching shaped-profile guide 57, and the shaped-profile cylinder 61 and the spacer stopper 63 having a fixing cylinder 64 with a conical external thread 65 for fixing the circular stopper plate 66 at a predetermined distance from the hollow cylindrical tube 58. The circular stopper plate 66 has an internal conical thread 67, into which the external conical thread 65 of the fixing cylinder 64 is screwed. The procedure of locking of the distal holes 54 of the cannulated intramedullary nail 53 with the shaped-profile canal 56 for inserting the matching shaped-profile guide 57 with the sensor 59 and the matching shaped-profile guide 57 does not differ from the above mentioned procedures described with reference to FIGS. 1, 2, 3, 4, and 5, in that, as far as functionality is concerned, only the embodiment of the guides, of the cannulated intramedullary nail, and of the stopping of the guide at the position of the distal hole are modified, which does not affect the procedure of locking of the cannulated nails as such. What separates the said embodiment from those described previously is that the stopping of the guide at the distal holes does not occur as a consequence of an internal locking of the guide at the position of the distal hole, but as a consequence of the external spacer stopper 63, which is made possible by the shaped-profile canal 56 of the cannulated intramedullary nail 53. The spacer stopper 63 may also be employed in the embodiment of FIG. 5, in that the manufacturer of the cannulated intramedullary nail may make only the groove 41 in the canal of the cannulated intramedullary nail 40, there being no necessity to also make the positional groove 42 for locking the guide at the position of the distal holes, since the said function is taken over by the external spacer stopper 63.

In the embodiments set forth hereinabove, spherical nipples 20 on the flat lamellae 19, spherical nipples 37 with a spring 38, and spacer locks 49 and specifically shaped internal profiles of the cannulated nails 40, 53 and of the guides 43, 57 have been described as means for positioning and securing the sensor 11 in the distal holes 16 of the cannulated intramedullary nail 9. Besides those described in the embodiments, however, other means and methods for positioning and fixing the sensor 11 in the distal holes 16 of the cannulated intramedullary nail 9 could clearly be envisioned by an expert in the field without departing from the scope of the present invention.

1-5. (canceled)

6. A system for facilitating a locking of distal holes in a cannulated intramedullary nail in surgery for repositioning a long-bone fracture, the system comprising:
A. a transmitter capable of representing an origin of a local coordinate system;
B. an internal guide including a first sensor, wherein the internal guide is capable of guiding the first sensor along the canal of the cannulated intramedullary nail to the distal holes and wherein the first sensor is capable of transmitting a position and rotation of the internal guide relative to the origin of the local coordinate system;
C. an extension including a second sensor, wherein the extension is adapted to allow use of drilling and screwing tools and wherein the second sensor is capable of transmitting a position and rotation of the extension relative to the origin of the local coordinate system; and
D. a real time rendering of a schematic image of the distal holes of the cannulated intramedullary nail and a schematic image of the extension to facilitate their alignment of along a common axis.

7. The system according to claim 6, wherein the internal guide further comprise an adaptation capable of being removably positioned and removably fixed in the distal holes of the cannulated intramedullary nail.

8. The system according to claim 7, wherein the internal guide further comprises a cylinder within which the first sensor is fixed and an adaptation capable of being removably positioned and removably fixed in the distal holes of the cannulated intramedullary nail.

9. The system according to claim 7, wherein the adaptation capable of being removably positioned and removably fixed in the distal holes of the cannulated intramedullary nail comprises one of nipples on flat lamellae, nipples biased with a spring, or spacer locks.

10. The system according to claim 7, wherein the canal of cannulated nail further comprises a shaped profile and the internal guide comprises a corresponding shape so as to be guidable, positionable and fixable inside the shaped profile.

11. A method for facilitating a locking of distal holes in a cannulated intramedullary nail in surgery for repositioning of a long-bone fracture, the method comprising:
A. transmitting a representation of an origin of a local coordinate system;
B. guiding an internal guide including a first sensor along the canal of the cannulated intramedullary nail to the distal holes;
C. transmitting a position and rotation of the internal guide relative to the origin of the local coordinate system;
D. providing an extension including a second sensor, wherein the extension is adapted to allow use of drilling and screwing tools and wherein the second sensor is capable of transmitting a position and rotation of the extension relative to the origin of the local coordinate system; and
E. rendering in real time a schematic image of the distal holes of the cannulated intramedullary nail and a schematic image of the extension to facilitate their alignment of along a common axis.

12. The method according to claim 11, wherein the internal guide further comprises an adaptation capable of being removably positioned and removably fixed in the distal holes of the cannulated intramedullary nail.

13. The method according to claim 11, wherein the internal guide further comprises a cylinder within which the first sensor is fixed and an adaptation capable of being removably positioned and removably fixed in the distal holes of the cannulated intramedullary nail.

14. The method according to claim 11, wherein the adaptation capable of being removably positioned and removably fixed in the distal holes of the cannulated intramedullary nail comprises one of nipples on flat lamellae, nipples biased with a spring, or spacer locks.

15. The method according to claim 11, wherein the canal of cannulated nail further comprises a shaped profile and the internal guide comprises a corresponding shape so as to be guidable, positionable and fixable inside the shaped profile.

16. A method for locking distal holes in a cannulated intramedullary nail, the method comprising the step of:
A. inserting an internal guide including a first sensor into the canal of the cannulated intramedullary nail, wherein the sensor is capable of transmitting in real time a position and rotation of the internal guide relative to an origin of a local coordinate system;
B. pushing the internal guide to the distal holes of the cannulated intramedullary nail;
C. positioning and fixing the internal guide in the distal holes of the cannulated intramedullary nail;
D. providing an extension adapted to allow a use of drilling and screwing tools and including a second sensor, wherein the second sensor is capable of transmitting in real time a position and rotation of the extension relative to the origin of the local coordinate system;
E. aligning a rendering of a schematic image of the distal holes of the cannulated intramedullary nail and a schematic image of the extension to align them along a common axis; and
F. drilling along the common axis using the extension.

17. The method according claim 16, wherein the internal guide further comprises an adaptation capable of being removably positioned and removably fixed in the distal holes of the cannulated intramedullary nail.

18. The method according claim 16, wherein the internal guide further comprises a cylinder within which the first sensor is fixed and an adaptation capable of being removably positioned and removably fixed in the distal holes of the cannulated intramedullary nail.

19. The method according claim 16, wherein the adaptation capable of being removably positioned and removably fixed in the distal holes of the cannulated intramedullary nail comprises one of nipples on flat lamellae, nipples biased with a spring, or spacer locks.

20. The method according to claim 16, wherein the canal of cannulated nail further comprises a shaped profile and the internal guide comprises a corresponding shape so as to be guidable, positionable and fixable inside the shaped profile.
